(19) United States
(12) Patent Application Publication
(10) Pub. No.: US 2022/0127337 A1
(43) Pub. Date: Apr. 28, 2022

(54) Anti-Viroporin Antibodies and Methods of Treating COVID-19

(71) Applicant: UNIVERSITY OF FLORIDA RESEARCH FOUNDATION, INCORPORATED, GAINESVILLE, FL (US)

(72) Inventors: Michael MCINTOSH, GAINESVILLE, FL (US); SUMITA BHADURI-MCINTOSH, GAINESVILLE, FL (US)

(21) Appl. No.: 17/512,027

(22) Filed: Oct. 27, 2021

Related U.S. Application Data
(60) Provisional application No. 63/105,986, filed on Oct. 27, 2020.

(51) Int. Cl. C07K 16/10; A61P 31/14; G01N 33/569

(52) U.S. Cl. CPC C07K 16/10; A61K 2039/505; G01N 33/56983; A61P 31/14

(57) ABSTRACT

Described are methods for generating human antibodies capable of blocking one or more functions of coronavirus viroporins. The methods can be used to generate therapeutic antibodies to treat or prevent coronavirus infection, including SARS-CoV-2 infection.

### CROSS-REFERENCE TO RELATED APPLICATIONS

This application claims the benefit of U.S. Provisional Application No. 63/105,986, filed, Oct. 27, 2020, which is incorporated herein by reference

### SEQUENCE LISTING

The Sequence Listing written in file 567056_T18331 SeqList.txt is 18 kilobytes in size, was created Oct. 21, 2021, and is hereby incorporated by reference

### BACKGROUND

COVID-19 is a highly communicable disease whose severity ranges from self-limited flu-like illness to respiratory failure, sepsis, and death. Even with a vaccine, severely ill and immunocompromised patients will remain vulnerable to COVID-19.

Antibody therapies and convalescent plasma transfusions have a long history of serving as a first line therapy in the treatment of emerging infections. For example, convalescent plasma therapy was employed during the original SARS outbreak in Asia in 2003, the 2014 Ebola virus disease outbreak in West Africa, and the Middle East Respiratory Syndrome (MERS) outbreak in 2015 (Chen L et al. Lancet Infect Dis 20, 398-400 (2020)). Convalescent plasma however provides a limited, perishable supply of therapeutic antibodies. In addition, plasma transfusions are not without significant risks, including exposure to uncharacterized extraneous human pathogens circulating in the population (Seghatchian J et al. Transfus Apher Sci 35, 189-196 (2006)).

Severe COVID-19 is not entirely dependent upon viral load, and is characterized by activation of the inflammatory pathway, often during a second phase of infection. Activation of the inflammatory pathway can lead to a cytokine storm, tissue damage, sepsis, respiratory failure, and possible death ((Liu Y et al., Lancet Infect Dis, (2020); Mehta P et al. Lancet 395, 1033-1034 (2020)). This inflammatory response, composed of IL-1β and other cytokines, results from assembly and activation of a multiprotein host machinery known as the inflammasome. The inflammasome can be activated in non-immune cells, such as airway epithelial cells, as well as immune cells. Several lines of evidence tie activation of the NLRP3 (NOD-, LRR- and pyrin domain-containing protein 3)-inflammasome to severe SARS-CoV-2 pathology.

Individuals with chronic diseases marked by pro-inflammatory conditions characterized by activation of the NLRP3 (NOD-, LRR- and pyrin domain-containing protein 3) inflammasome, such as atherosclerosis, obesity, and diabetes, are known to be at significantly increased risk for severe disease (Wu Z et al. JAMA 323(13):1239-1242 (2020); Alhawiti N M et al. Curr Drug Targets 18, 1095-1103 (2017); Chen W et al. Inflamm Res 66, 157-166 (2017); Jin Y et al. J Am Heart Assoc 8, e012219 (2019); Rheinheimer J et al. Metabolism 74, 1-9 (2017); Stienstra R et al. Proc Natl Acad Sci USA 108, 15324-15329 (2011); Dixit V D. Diabetes 62, 22-24 (2013); Grant R W et al. Front Immunol 4, 50 (2013); Zhang X et al. Antioxid Redox Signal 22, 848-870 (2015); Richardson S et al., JAMA. 323(20): 2052-2059 (2020); Yang Jet al. Int J Infect Dis 94, 91-95 (2020)). SARS-CoV-2 is also linked to Multisystem Inflammatory Syndrome in Children (MIS-C) (Feldstein LR et al., Multisystem Inflammatory Syndrome in U.S. Children and Adolescents. N Engl J Med 383, 334-346 (2020) and Multisystem Inflammatory Syndrome in Adults (MIS-A) (Morris, S. B. et al. Morbidity and Mortality Weekly Report (MMWR) 69, 1450-1456 (2020)).

New antiviral therapies are needed to reduce spread of coronavirus, such as SARS-CoV-2, and to treat those exposed to or infected by the virus.

### SUMMARY

Described are methods for generating human antibodies capable of blocking one or more functions of coronavirus viroporins. The coronavirus viroporins can be, but are not limited to, SARS-CoV-2 viroporins, ORF3a and E. The methods can be used to generate therapeutic antibodies to treat or prevent coronavirus infection, including SARS-CoV-2 infection.

In some embodiments, methods of generating human antibodies capable of blocking one or more functions of coronavirus viroporins are described comprising: obtaining primary human B-lymphocytes (B cells) from one or more subjects that have recovered from coronavirus infection, identifying one or more primary human B-lymphocytes producing anti-viroporin antibodies, immortalizing the one or more B-lymphocytes identified as producing anti-viroporin antibodies, and producing anti-viroporin antibodies from the one or more immortalized B-lymphocytes. The coronavirus infection can be, but is not limited to, COVID-19.

In some embodiments, methods of generating human antibodies capable of blocking one or more functions of coronavirus viroporins are described comprising: obtaining one or more primary human B-lymphocytes from one or more subjects that have recovered from coronavirus infection, identifying one or more primary human B-lymphocytes producing one or more anti-viroporin antibodies, generating one or more recombinant nucleic acids encoding the one or more anti-viroporin antibodies from the one or more B-lymphocytes identified as producing the one or more anti-viroporin antibodies, generating one or more cell lines expressing the one or more recombinant nucleic acids, and producing anti-viroporin antibodies from the one or more cell lines expressing the one or more recombinant nucleic acids. The coronavirus infection can be, but is not limited to, COVID-19. In some embodiments, the coronavirus viroporin is a SARS-CoV-2 viroporin. In some embodiments, the one or more primary human B-lymphocytes identified as producing the one or more anti-viroporin antibody are immortalized.

In some embodiments, methods of generating human antibodies capable of blocking one or more functions of coronavirus viroporins are described comprise: obtaining one or more primary human B-lymphocytes from one or more subjects that have recovered from coronavirus infection, immortalizing the one or more primary human B-lymphocytes, identifying one or more immortalized human B-lymphocytes producing anti-viroporin antibodies, generating one or more recombinant nucleic acids encoding the one or more anti-viroporin antibodies from the one or more immortalized B-lymphocyte identified as producing the one or more anti-viroporin antibodies, generating one or more cell lines expressing the one or more recombinant nucleic acids, and producing anti-viroporin antibodies from the one or more cell line expressing the one or more recombinant nucleic acids. The coronavirus infection can be, but is not limited to, COVID-19. In some embodiments, the coronavirus viroporin is a SARS-CoV-2 viroporin.

In some embodiments, the methods of generating human antibodies capable of blocking one or more functions of coronavirus viroporins are high-throughput. In some embodiments, the methods of generating human antibodies capable of blocking one or more functions of SARS-CoV-2 viroporins are high-throughput.

Also described are recombinant engineering programs to manufacture human-derived polyclonal and monoclonal antibodies (mAbs) to serve as coronavirus immunotherapeutics. The coronavirus immunotherapeutics can be, but are not limited to, SARS-CoV-2 immunotherapeutics.

The anti-viroporin antibodies can be administered to a subject to treat or prevent coronavirus disease. In some embodiments, the anti-viroporin antibodies are administered to a subject to treat or prevent one or more symptoms associated with coronavirus disease. In some embodiments, the anti-viroporin antibodies are administered to a subject to treat or prevent an inflammatory response in a subject having coronavirus disease. In some embodiments, the subject suffers from coronavirus disease or severe coronavirus disease. The coronavirus disease can be, but is not limited to COVID-19.

The anti-viroporin antibodies can be administered to a subject to treat or prevent coronavirus infection. In some embodiments, the anti-viroporin antibodies are administered to a subject to treat or prevent one or more symptoms caused by or associated with coronavirus infection. In some embodiments, the anti-viroporin antibodies are administered to a subject to treat or prevent an inflammatory response caused by coronavirus infection. The coronavirus infection can be, but is not limited to SARS-CoV-2 infection.

In some embodiments, the anti-viroporin antibodies are administered to a subject to treat or suppress virus-triggered inflammation associated with SARS-CoV-2 infection or COVID-19. The virus-triggered inflammation can be, but is not limited to, a cytokine storm, an inflammasome-associate response, an IL-IP-associated response, or an NLRP3-associated response, or combinations thereof. In some embodiments, the anti-viroporin antibodies are administered to a subject to treat or prevent Multisystem Inflammatory Syndrome in Children or Adults (MIS-C and MIS-A).

In some embodiments, the subject has defective type I interferon immunity, has a chronic disease marked by pro-inflammatory conditions characterized by activation of the NLRP3 (NOD-, LRR- and pyrin domain-containing protein 3) inflammasome, suffers from a metabolic disturbance, is immunocompromised, has diabetes, has atherosclerosis, and/or is obese. The metabolic disturbance can be, but is not limited to hypokalemia.

The anti-viroporin antibodies can be administered to a subject to block or inhibit coronavirus release from an infected cell or to block or inhibit spread of the coronavirus. The coronavirus can be, but is not limited to, SARS-CoV-2.

The anti-viroporin antibodies can be used in combination with one or more additional therapies. The one or more additional therapies can be, but are not limited to, additional antibody therapies, monoclonal antibody therapy, convalescent plasma therapy, anti-SARS-CoV-2 Spike protein antibody therapy, anti-inflammation therapy, inflammasome therapy, remdesivir, dexamethasone, baricitinib, tofacitinib, tocilizumab, and sarilumab. Anti-inflammation therapy can be, but is not limited to, anakinra therapy or a similar IL-1 blocking therapy.

In some embodiments, the anti-viroporin antibodies are used in diagnosis of SARS-CoV-2 infection. The anti-viroporin antibodies can be used to detect SARS-CoV-2 in a sample.

### BRIEF DESCRIPTION OF THE DRAWINGS

FIG. 1A-C. ORF3a primes and activates the inflammasome, causing cell death. HEK-293T cells or A549 cells were transfected with FLAG-tagged ORF3a or empty vector (EV) and harvested after for 24 hours for immunoblotting with indicated antibodies (A, C), reverse transcriptase-PCR for IL-1β mRNA abundance (B). A and C: HEK-293T in left panel, A549 in right panel. All experiments were performed three times.

FIG. 1D-E. ORF3a primes and activates the inflammasome, causing cell death. Cells were treated as described in FIG. 1A-C and harvested after 24 hours for ChIP-PCR to quantify relative enrichment of NFkB p65 at the IL-1β promoter using anti-p65 antibodies (black bar) or control IgG (white bar) (D) or immunoblotting with indicated antibodies (E). Error bars in D represent SEM. D and E: A549 cells. All experiments were performed three times.

FIG. 1F-G. ORF3a primes and activates the inflammasome, causing cell death. Cells were treated as described in FIG. 1A-C and harvested after 24 hours for immunoblotting with the indicated antibodies (F and G). HEK-293T in left panel, A549 in right panel. All experiments were performed three times.

FIG. 1H. ORF3a primes and activates the inflammasome, causing cell death. Cells were treated as described in FIG. 1A-C and harvested after 24 hours. Unfixed cells were stained with propidium iodide followed by flow cytometry to enumerate percent dead cells. All experiments were performed three times.

FIG. 2A-B. ORF3a activates the NEK7-NLRP3 inflammasome via ASC-dependent and independent modes. (A) Cell lysates of FLAG-ORF3a- or EV-transfected HEK-293T (left) or A549 cells (right) were immunoblotted with the indicated antibodies. (B) HEK-293T (left) or A549 cells (right) were co-transfected with FLAG-ORF3a and either control siRNA or NLRP3 siRNA (B) for 24 hours prior to immunoblotting with indicated antibodies. Experiments were performed at least thrice.

FIG. 2C-D. ORF3a activates the NEK7-NLRP3 inflammasome via ASC-dependent and independent modes. (C) HEK 293T (left) or A549 cells (right) were transfected with EV or FLAG-ORF3a and exposed to MCC950 for 24 hours prior to immunoblotting. (D) HEK-293T (left) or A549 cells (right) were co-transfected with FLAG-ORF3a and either control siRNA or NEK7 siRNA for 24 hours prior to immunoblotting with indicated antibodies. Experiments were performed at least thrice.

FIG. 2E-F. ORF3a activates the NEK7-NLRP3 inflammasome via ASC-dependent and independent modes. (E) Cell lysates were immunoblotted with the indicated antibodies. (F) A549 cells were co-transfected with FLAG-ORF3a and either control siRNA or ASC siRNA for 24 hours prior to immunoblotting with indicated antibodies. Experiments were performed at least thrice.

FIG. 3A-B. ORF3a-mediated activation of NLRP3 inflammasome requires K+ efflux. (A) FLAG-ORF3a plasmid or EV were introduced into A549 cells. After 20 hours, cells were left in normal medium (N) or exposed to medium with high K+(50mM; to block K+ efflux; High). Cells were harvested 4 hours later, and extracts immunoblotted with the indicated antibodies. (B) A549 cells transfected with Flag-ORF3a were exposed to the indicated potassium channel inhibitors for 24 hours prior to immunoblotting with different antibodies. Experiments were performed twice.

FIG. 3C-D. ORF3a-mediated activation of NLRP3 inflammasome requires K+ efflux. FLAG-ORF3a plasmid was introduced into A549 (C) and HEK293T (D) cells. After 20 hours, cells were left in normal medium (N) or exposed to medium with high K+(High). Cells were harvested 4 hours later, and extracts immunoblotted (Input) or immunoprecipitated with control IgG or anti-NEK7 antibodies followed by immunoblotting with indicated the antibodies. Input represents 5% of sample. Experiments were performed twice.

FIG. 4A. ORF3a residues required for inflammasome activation are conserved in SARS-CoV-2 isolates across continents. ORF3a/ORF3 viroporin from SARS-like betacoronaviruses, including temporally and geographically distinct isolates from the COVID-19 pandemic and diverse species isolates dating back to the original SARS pandemic of 2003, were aligned in CLUSTAL Omega using EMBL-EBI Server Tools (https://www.ebi.ac.uk/Tools/services/web_clustalo/toolform.ebi). Selected isolates displaying the most diversity are shown from positions 81 to 160 of ORF3a/ORF3. Position of conserved cysteine residues previously identified in SARS-CoV as critical to K+ ion channel formation are outlined. Newly divergent residues (121 and 153) conserved across SARS-CoV-2 isolates are in bold.

| Coronavirus isolate | SEQ ID NO. |
|---|---|
| SARS-CoV-2 Wuhan 2019 | 1 |
| USA CA January 2020 | 2 |
| USA NY March 2020 | 3 |
| Russia March 2020 | 4 |
| Sri Lanka March 2020 | 5 |
| Greece Ma2 2020 | 6 |
| Saudi Arabia March 2020 | 7 |
| Spain March 2020 | 8 |
| France March 2020 | 9 |
| Brazil March 2020 | 10 |
| USA FL April 2020 | 11 |
| Australia April 2020 | 12 |
| India April 2020 | 13 |
| Bangladesh June 2020 | 14 |
| SARS-CoV-2 NY Tiger | 15 |
| Bat CoV 2017 | 16 |
| Bat CoV 2013 | 17 |
| Bat SARS WIV16 2013 | 18 |
| Bat SARS Rs4231 2013 | 19 |
| Bat SARS HKU3-9 2009 | 20 |
| Bat CoV 2008 | 21 |
| Bat SARS HKU3-3 2005 | 22 |
| SARS-CoV 2003 | 23 |

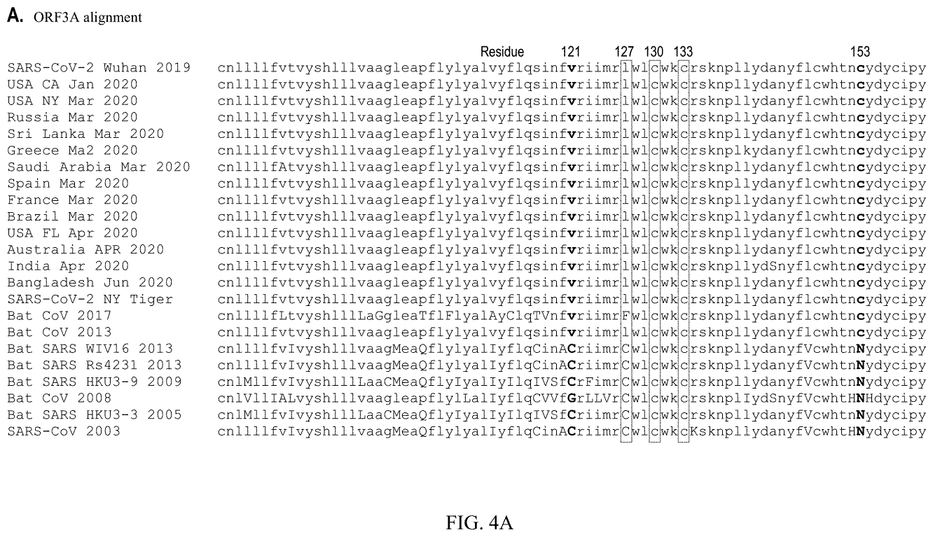

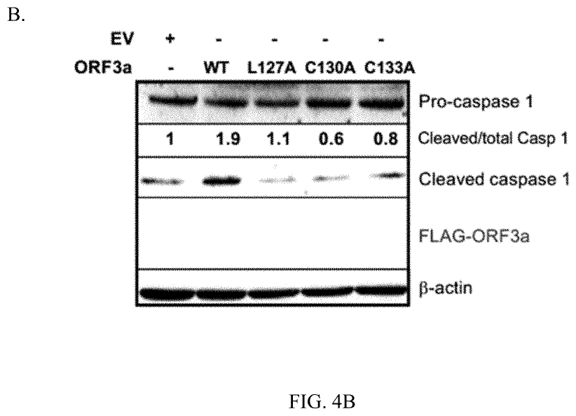

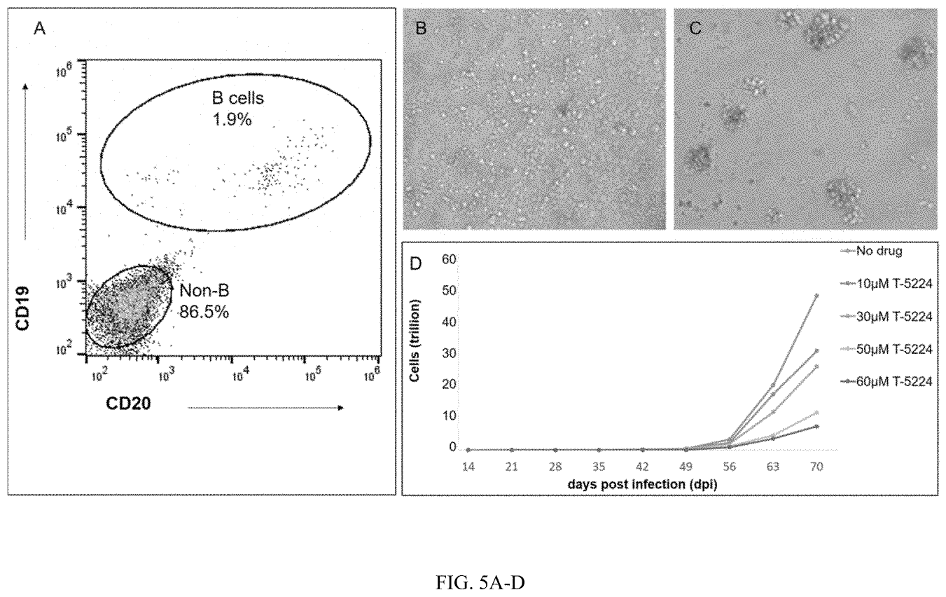

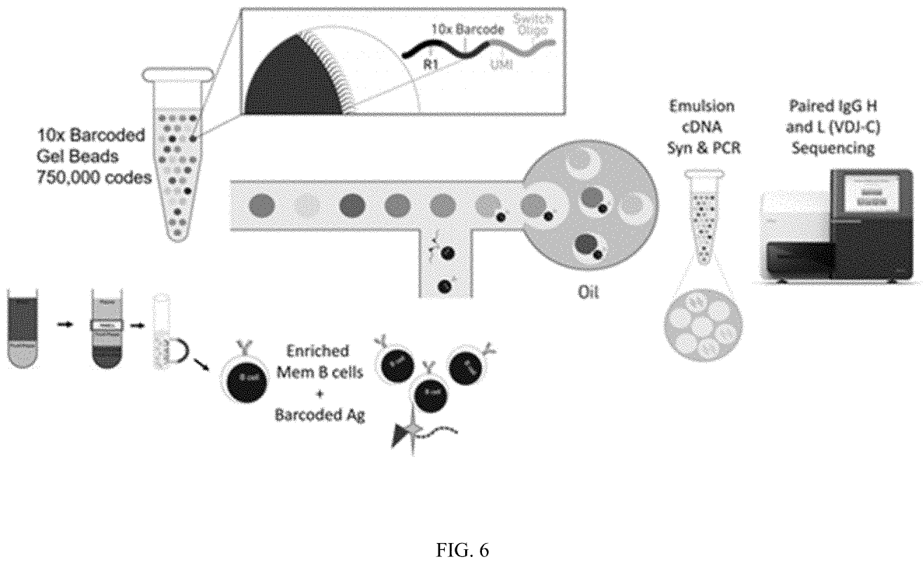

FIG. 4B. ORF3a residues required for inflammasome activation are conserved in SARS-CoV-2 isolates across continents. A549 cells were transfected with EV, wild-type FLAG-ORF3a (WT), or FLAG-ORF3a mutants (L127A, C130A, and C133A). Cells were harvested 24 hours later and immunoblotted with the indicated antibodies.

FIG. 5A-D. Isolation of primary B cells and establishment of B cell lines. (A) Peripheral blood mononuclear cells (PBMC) from a healthy human donor displayed 1.9% CD19 and CD20 positive B cells. CD19 and CD20 represent B cell specific markers. (B and C) PBMC were infected with EBV in the presence of the T cell inhibitor FK506. (B) Phase contrast image of PBMC at the time of EBV exposure. (C) Transformed proliferating B cell clusters 1-week post EBV exposure. (D) Long-term expansion of B cell lines in the presence or absence of an AP-1 inhibitor (T-5224).

FIG. 6. 10× Genomic high-throughput single cell partitioning and LIBRA-Seq (linking B cell receptor to antigen specificity through sequencing). Unique barcoded gel beads associate with single cells traveling through microfluidic channels that are then extruded into single bubbles in an aqueous oil emulsion allowing for clonal oligo dT-primed cDNA synthesis and amplification for next-generation sequencing. LIBRA-Seq tags antigens of interest (Orf3a and E pep-tides) with oligonucleotide barcodes allowing B cells bound by surface Ig to an antigen to have two simultaneous bar codes (a first to identify the cell and the second its bound antigen). This enables massively paralleled sequencing of antibody heavy and light genes (VDJ-C regions) linked by the antigen binding specificity of each individual B cell. The process is enhanced by magnetic bead enrichment of CD19+, CD27+memory B cells from peripheral blood and/or enriching for B cells bound to select oligo barcoded antigens of interest by fluorescence activated cell sorting using fluorophore (Orange Star) tagged and barcoded antigens (adapted with permission of 10X Genomics Inc.).

### DETAILED DESCRIPTION

Described are methods of identifying and manufacturing antibodies that target coronavirus virus-encoded viroporins that may be triggers of the inflammatory cascade. The described methods identify and produce authentic human antibodies with near unlimited production capacity from a controlled source through B cell immortalization and recombinant engineering.

Viroporins are virus-encoded proteins that form pores that facilitate ion transport across cell membranes. Viroporin ion pore activity is believed to facilitate virus release from infected host cells (DeDiego M L et al. J Virol 81, 1701-1713 (2007); DeDiego M L et al. Virus Res 194, 124-137 (2014); and Dediego M L et al. Virology 376, 379-389 (2008)). Viroporins from SARS-CoV have been found to activate the NLRP3 inflammasome (Farag N S et al. Int J Biochem Cell Biol 122, 105738 (2020); Castano-Rodriguez C. et al. mBio 9 (2018); Chen IY et al. Front Microbiol 10, 50 (2019); Fung S Y et al. Emerg Microbes Infect 9, 558-570 (2020); DeDiego M L et al. Virology 376, 379-389 (2008)).

We have found that the highly conserved surface channels encoded by SARS-CoV-2 ORF3a and E (viroporins) prime and trigger the NLRP3 inflammasome, resulting in Interleukin 1 beta (IL-1β) expression and activation at the apex of the inflammatory cascade. The viroporin encoded by ORF3a is an essential non-structural protein expressed early from a sub-genomic message that is required for release of SARS-CoV virions (Lu W et al., Proc Natl Acad Sci USA 103, 12540-12545 (2006)). E is an envelope protein that also forms ion channels.

### I. Definitions

“Inflammasomes” are a group of cytosolic protein complexes that are formed to mediate host immune responses to microbial infection and cellular damage. The activation of inflammasomes is a major inflammatory pathway. Assembly of an inflammasome triggers proteolytic cleavage of dormant procaspase-1 into active caspase-1, which converts the cytokine precursors pro-IL-lb and pro-IL-18 into mature and biologically active IL-1β and IL18. Mature IL-1β is a potent proinflammatory mediator in many immune reactions. Active caspase-1 induces a proinflammatory form of cell death known as pyroptosis. Inflammasomes are activated by pattern-recognition receptors (PRRs) in response to pathogen-associated molecular patterns (PAMPs) or damage-associated molecular patterns (DAMPs). Five PRRs, NLRP1, NLRP3, NLRC4 (nucleotide-binding oligomerization domain (NOD), leucine-rich repeat (LRR)-containing proteins (NLR) family members), Pyrin, and AIM2 (absent-in-melanoma 2), have been shown to form inflammasomes.

The NLRP3 inflammasome is important for host immune defenses against bacterial, fungal, and viral infections. NLPR3 has also been linked to the pathogenesis of several inflammatory disorders when dysregulated, including cryopyrin-associated periodic syndromes (CAPS), Alzheimer's disease, diabetes, gout, autoinflammatory diseases, and atherosclerosis.

A “nucleic acid” includes both RNA and DNA. RNA and DNA include, but are not limited to, cDNA, genomic DNA, plasmid DNA, RNA, mRNA, condensed nucleic acid, nucleic acid formulated with cationic lipids, and nucleic acid formulated with peptides or cationic polymers. Nucleic acid also includes modified RNA or DNA.

An “expression vector” refers to a nucleic acid (e.g., RNA or DNA) encoding an expression product (e.g., peptide(s) (i.e., polypeptide(s) or protein(s)) or RNA), such as an antibody. An expression vector may be, but is not limited to, a virus, a modified virus, a recombinant virus, an attenuated virus, a plasmid, a linear DNA molecule, or an mRNA. An expression vector is capable of expressing one or more polypeptides in a cell, such as mammalian cell. The expression vector may comprise one or more sequences necessary for expression of the encoded expression product. A variety of sequences can be incorporated into an expression vector to alter expression of the coding sequence. The expression vector may comprise one or more of: a 5′ untranslated region (5′ UTR), an enhancer, a promoter, an intron, a 3′ untranslated region (3′ UTR), a terminator, and a polyA signal operably linked to the DNA coding sequence.

The term “plasmid” refers to a nucleic acid that includes at least one sequence encoding a polypeptide (such as a transdifferentiation determinant) that is capable of being expressed in a cell. A plasmid can be a closed circular DNA molecule. A variety of sequences can be incorporated into a plasmid to alter expression of the coding sequence or to facilitate replication of the plasmid in a cell. Sequences can be used that influence transcription, stability of a messenger RNA (mRNA), RNA processing, or efficiency of translation. Such sequences include, but are not limited to, 5′ untranslated region (5′ UTR), promoter, introns, and 3′ untranslated region (3′ UTR). Plasmids can be manufactured in large scale quantities and/or in high yield. Plasmids can further be manufacture using cGMP manufacturing. Plasmids can be transformed into bacteria, such as E. coli. Plasmids can be transformed into bacteria, including, but not limited to, Chinese hamster ovary (CHO) cells, hybridoma cells, myeloma cells, NSO murine myeloma cells, and PER.C6 human cells. The DNA plasmids are can be formulated to be safe and effective for injection into a mammalian subject.

A “promoter” is a DNA regulatory region capable of binding an RNA polymerase in a cell (e.g., directly or through other promoter-bound proteins or substances) and initiating transcription of a coding sequence. A promoter may comprise one or more additional regions or elements that influence transcription initiation rate, including, but not limited to, enhancers. A promoter can be, but is not limited to, a constitutively active promoter, a conditional promoter, an inducible promoter, or a cell-type specific promoter. Examples of promoters can be found, for example, in WO 2013/176772. The promoter can be, but is not limited to, CMV promoter, Igκ promoter, mPGK, SV40 promoter, β-actin promoter (such as, but not limited to a human or chicken β-actin promoter), α-actin promoter, SRα promoter, herpes thymidine kinase promoter, herpes simplex virus (HSV) promoter, mouse mammary tumor virus long terminal repeat (LTR) promoter, adenovirus major late promoter (Ad MLP), rous sarcoma virus (RSV) promoter, and EF1α promoter. The CMV promoter can be, but is not limited to, CMV immediate early promoter, human CMV promoter, mouse CMV promoter, and simian CMV promoter. The promoter can also be a hybrid promoter. Hybrid promoters include, but are not limited to, CAG promoter.

A “heterologous” sequence is a sequence which is not normally present in a cell, genome or gene in the genetic context in which the sequence is currently found. For example, a heterologous sequence can be a coding sequence linked to a different promoter sequence relative to the native coding sequence. A heterologous sequence can differ from its corresponding native sequence in having one or more introns removed. A heterologous sequence can also be present in the context of an expression vector, such as, but not limited to, a plasmid or viral vector.

A “complementarity determining region,” and “CDR,” refers to non-contiguous sequences of amino acids within antibody variable regions, which confer antigen specificity and binding affinity. In general, there are three (3) CDRs in each heavy chain variable region (CDR-H1, CDR-H2, CDR-H3) and three (3) CDRs in each light chain variable region (CDR-L1, CDR-L2, CDR-L3).

A “pharmacologically effective amount,” “therapeutically effective amount,” “effective amount,” or “effective dose” refers to that amount of an agent (e.g., antibody) to produce the intended pharmacological, therapeutic or preventive result.

### II. Anti-Coronavirus Viroporin Antibodies

Described are methods of generating anti-coronavirus viroporin antibodies from B lymphocytes isolated from subjects who have recovered from coronavirus infection. B lymphocytes identified as producing anti-viroporin antibodies can be immortalized, thereby providing a source of the anti-viroporin antibodies. Alternatively, anti-viroporin antibody nucleic acid coding sequences can be cloned from B lymphocytes identified as producing anti-viroporin antibodies. Recombinant anti-viroporin antibody nucleic acid sequences can then be used to produce anti-viroporin antibodies in cell types suitable for industrial scale antibody synthesis.

A. Isolating B lymphocytes

B lymphocytes (B cells) are obtained from healthy subjects who have recovered from coronavirus infection, such as SARS-CoV-2 infection or COVID-19, the illness caused by SARS-CoV-2. B cells from such subjects can be obtained by methods known in the art. In some embodiments, peripheral blood mononuclear cells (PBMCs) are obtained from the subject and B lymphocytes are then purified or isolated from the PBMCs. B lymphocytes can be isolated or purified from PBMCs by cell sorting and selecting cells positive for B lymphocyte surface markers such as CD19 and CD20.

### B. Immortalizing Primary B Lymphocytes

Primary B lymphocytes isolated from a convalescent subject may be immortalized using methods known in the art for immortalizing immune cells such as B lymphocytes. In some embodiments, the primary B lymphocytes are immortalized by infection with Epstein-Barr virus to generate immortalized lymphoblastoid cell lines (LCL). In other embodiments, B cells may be immortalized by other methods including transduction with a lentivirus capable of delivering expression constructs for the human telomerase gene alternate oncogene known to transform or immortalize primary cells. In some embodiments, the immortalized B cells can be used as a source of antibodies. In some embodiments, the immortalized B cells can be used as a source of nucleic acid sequences encoding antibodies or antigen binding fragments thereof that have affinity for viroporins.

### C. Selecting Primary or Immortalized B Lymphocytes Having Affinity for Viroporins

The isolated and optionally immortalized B lymphocytes are screened for B lymphocytes having affinity for viroporins using methods known in the art. Suitable methods for identifying B lymphocytes having affinity for viroporins include, but are not limited to, ELISA, cell sorting, and 10× Genomics Inc. platform and LIBRA-seq (see example 6). LIBRA-seq is a technology for high-throughput mapping of paired heavy- and light-chain B cell receptor sequences to their cognate antigen specificities (Setliff I et al. Cell179, 1636-1646 e1615 (2019). 10× Genomics Inc. platform and LIBRA-seq is used to rapidly identify B lymphocytes with surface IgG having affinity for viroporins or to a recombinant peptide (labeled DNA-barcoded antigens) from coronavirus viroporins. Using 10× Genomics Inc. platform and LIBRA-seq, it is possible to massively parallel sequence many individual B cell surface IgG genes encoding antibodies with viroporin-specific binding.

### D. Cloning Anti-Viroporin Antibodies

After B lymphocytes having surface IgG with affinity for coronavirus viroporins are identified, the anti-viroporin antibody genes encoding the anti-viroporin antibodies, or antigen binding fragments thereof, can be cloned and sequenced. In some embodiments, heavy and light chain (Igλ or Igκ) VDJ sequences are cloned and/or sequenced. In some embodiments, heavy and light chain (Igλ or Igκ) VDJ sequences are assembled using 10× Genomics Cell Ranger Analysis Pipelines. The heavy and light chain variable sequences can be synthesized and cloned into expression vectors. In some embodiments, the heavy and light chain variable sequences are synthesized and cloned separately into expression vectors containing human Ig constant regions for the heavy chain and light chains.

In some embodiments, the full antibody IgG coding sequences (heavy and light chains) are cloned. In some embodiments, antigen binding fragments of the antibodies are cloned. Antigen binding fragments may comprise heavy and light chain variable regions or heavy and light chain complementarity-determining regions (CDR) sequences. The cloned sequences can be used to make recombinant nucleic acids encoding the recombinant antibodies or antigen binding fragments thereof. The recombinant nucleic acids can then be used to make antibodies or antigen binding fragments. The heavy and light chains sequences (e.g., full length sequences, variable domain sequences, or CDR sequences) can be cloned into a single expression vector or into separate expression vectors for heavy and light chain sequences. The heavy chain variable regions and light chain variable regions or CDRs may be formatted into a structure of a natural antibody or functional fragment or equivalent thereof. The cloned sequences may be formatted in a full length antibody, a (Fab′)2 fragment, a Fab fragment, or equivalent thereof (such as a single change variable fragment (scFv)). The antibody may be an IgG₁, IgG₂, IgG₃, IgG₄, IgM, IgA, IgE or IgD or a modified variant thereof.

In some embodiments, a cloned heavy or light chain variable sequence is operably linked to a heterologous heavy or light chain constant region to form a nucleic acid encoding a full length heavy or light chain. In some embodiments, cloned heavy or light chain CDR sequences are inserted into a nucleic acid encoding a heterologous full length heavy or light chain such that the recombinant nucleic acid encodes a full length antibody sequence having the cloned CDR sequences. In some embodiments, cloned heavy or light chain CDR sequences are inserted into a nucleic acid encoding a heterologous heavy or light chain variable region such that the recombinant nucleic acid encodes a heavy or light chain variable region having the cloned CDR sequences.

A recombinant nucleic acid encoding an anti-viroporin antibody, or antigen binding fragment thereof, can be operably linked to one or more sequences necessary for expression of the encoded expression product. A variety of sequences can be incorporated into an expression vector to alter expression of the coding sequence. The expression vector may comprise one or more of: a 5′ untranslated region (5′ UTR), an enhancer, a promoter, an intron, a 3′ untranslated region (3′ UTR), a terminator, and a polyA signal operably linked to the DNA coding sequence.

### E. Manufacturing

In some embodiments, the anti-viroporin antibodies are produced from activated immortalized B lymphocytes.

In some embodiments, the anti-viroporin antibodies, or antigen binding fragments thereof, are produced by transfection of one or more host cells with one or more expression vectors encoding the antibodies, or antigen binding fragments thereof. Suitable host cells or cell lines for the expression of the antibodies, or antigen binding fragments thereof, include mammalian cells such as NS0 cells, Sp2/0 cells, CHO (e.g., DG44) cells, COS cells, HEK (e.g., HEK293T) cells, fibroblast (e.g., 3T3) cells, and myeloma cells. In some embodiments, the host cell is a human cell line.

Cell culture media from transfected cells can be analyzed for the expression of antibodies (such as IgG) by anti-human IgG ELISA using plates coated with cognate recombinant antigens derived from ORF3a or E. Antibodies (or antigen binding fragments thereof) from the cell culture media can also be analyzed for their ability to recognize viroporins in cells expressing heterologous viroporin.

The present invention also encompasses a cell line transfected with a recombinant nucleic acid encoding an anti-viroporin antibody, or antigen binding fragments thereof. The cell can be, but is not limited to, a NS0 cell, a Sp2/0 cell, a CHO (e.g., DG44) cell, a COS cell, a HEK (e.g., HEK293T) cell, a fibroblast (e.g., 3T3) cell, and a myeloma cell.

III. Methods of using the antibodies

The identified anti-viroporin antibodies can be used to block one or more functions of coronavirus viroporins. Blocking one or more functions of a coronavirus viroporin comprises inhibiting interaction of the viroporin with another protein or inhibiting an activity of the viroporin as measured in the presence of the antibody compared to the interaction or activity of the viroporin in the absence of the antibody.

The identified anti-viroporin antibodies can be used to block or inhibit coronavirus release from an infected cell or to block or inhibit spread of the coronavirus.

The identified anti-viroporin antibodies can be used to treat or prevent coronavirus infection. Treating or preventing coronavirus infection comprises inhibiting or reducing coronavirus replication, secretion, infectivity, or reducing or inhibiting one or more symptoms associated with coronavirus infection. The one or more symptoms associated with coronavirus infection can be independently selected from the list consisting of: an inflammatory response, a cytokine storm, an inflammasome-associate response, an IL-1β-associated response, or an NLRP3-associated response.

The identified anti-viroporin antibodies can be administered to a subject to treat or prevent Multi system Inflammatory Syndrome in Children or Adults (MIS-C and MIS-A).

The identified anti-viroporin antibodies can be used to treat or prevent one or more symptoms in a subject, wherein the subject has defective type I interferon immunity, has a chronic disease marked by pro-inflammatory conditions characterized by activation of the NLRP3 (NOD-, LRR- and pyrin domain-containing protein 3) inflammasome, suffers from a metabolic disturbance, is immunocompromised, has diabetes, has atherosclerosis, and/or is obese. The metabolic disturbance can be, but is not limited to hypokalemia.

The terms “treat,” “treatment,” and the like, mean the methods or steps taken to provide relief from or alleviation of the number, severity, and/or frequency of one or more symptoms of a disease or condition in a subject. Treating generally refers to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term treatment can include: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease and/or its symptoms or conditions. Treating can refer to both therapeutic treatment alone, prophylactic treatment alone, or both therapeutic and prophylactic treatment. Those in need of treatment (subjects in need thereof) can include those already with coronavirus infection that or those in which infection is to be prevented. Treating can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the inflammation without preventing viral replication.

### IV. Listing of Embodiments

1. A method of generating human antibodies capable of blocking one or more functions of a coronavirus viroporin comprising: (a) obtaining primary human B-lymphocytes from a subject that has recovered from coronavirus infection; (b) identifying one or more primary human B-lymphocytes having surface IgG having affinity for the viroporin; (c) immortalizing the one or more B-lymphocytes identified as having surface IgG having affinity for the viroporin; and (d) producing anti-viroporin antibodies from the one or more immortalized B-lymphocytes.

2. A method of generating human antibodies capable of blocking one or more functions of a coronavirus viroporin comprising: (a) obtaining primary human B-lymphocytes from a subject that has recovered from coronavirus infection; (b) immortalizing the B-lymphocytes from step (a); (c) identifying one or more immortalized B-lymphocytes having surface IgG having affinity for the viroporin; and (d) producing anti-viroporin antibodies from the one or more immortalized B-lymphocytes identified as having surface IgG having affinity for the viroporin.

3. A method of generating human antibodies capable of blocking one or more functions of a coronavirus viroporin comprising: (a) obtaining primary human B-lymphocytes from a subject that has recovered from coronavirus infection; (b) identifying one or more primary human B-lymphocytes having surface IgG having affinity for the viroporin, (c) generating one or more recombinant nucleic acids encoding anti-viroporin antibodies or antigen binding fragments thereof from the one or more B-lymphocytes identified having surface IgG having affinity for the viroporin; (d) generating one or more cell lines expressing the one or more recombinant nucleic acids; and (e) producing anti-viroporin antibodies from the one or more cell lines expressing the one or more recombinant nucleic acids.

4. A method of generating human antibodies capable of blocking one or more functions of a coronavirus viroporin comprising: (a) obtaining primary human B-lymphocytes from a subject that has recovered from coronavirus infection; (b) immortalizing the B-lymphocytes from step (a); (c) identifying one or more immortalized B-lymphocytes having surface IgG having affinity for the viroporin; (d) generating one or more recombinant nucleic acids encoding anti-viroporin antibodies or antigen binding fragments thereof from the one or more immortalized B-lymphocytes identified as having surface IgG having affinity for the viroporin; (e) generating one or more cell lines expressing the one or more recombinant nucleic acids; and (f) producing anti-viroporin antibodies from the one or more cell lines expressing the one or more recombinant nucleic acids.

5. The method of any one of embodiments 1-4 wherein the coronavirus is a betacoronavirus.

6. The method of embodiment 5, wherein the betacoronavirus is SARS-CoV-2.

7. The method of any one of embodiments 1-6, wherein the viroporin is an ORF3a protein or an E protein.

8. A method of treating or preventing coronavirus infection in a subject comprising administering to the subject an effective amount of the antibodies of any one of embodiments 1-7.

9. The method of embodiment 8, wherein treating coronavirus infection comprises treating or preventing one or more symptoms associated with coronavirus disease or infection.

10. The method of embodiment 9, wherein the symptom associated with coronavirus disease or infection comprises an inflammatory response.

11. The method of embodiment 10, wherein the inflammatory response comprises a cytokine storm, an inflammasome-associate response, an IL-IP-associated response, an NLRP3-associated response, Multisystem Inflammatory Syndrome in Children, or Multisystem Inflammatory Syndrome in Adults.

12. The method of any one of embodiments 1-11, wherein the subject has defective type I interferon immunity, has a chronic disease marked by pro-inflammatory conditions characterized by activation of the NLRP3 inflammasome, suffers from a metabolic disturbance, is immunocompromised, has diabetes, has atherosclerosis, and/or is obese.

13. The method of embodiment 12, wherein the metabolic disturbance comprises hypokalemia.

14. The method of embodiment 9, wherein the coronavirus disease is COVID-19.

15. The method of any one of embodiments 1-14, wherein the coronavirus infection is SARS-CoV-2 infection.

16. The method of any one of embodiments 1-15, further comprising administering to the subject an effective dose of an additional anti-coronavirus antibody therapy, a convalescent plasma therapy, an anti-inflammation therapy, or an inflammasome therapy.

17. A method of diagnosing SARS-CoV-2 infection comprising analyzing (a) obtaining a sample from a subject; and (b) using an antibody of any one of embodiments 1-7 to detect the presence or absence of a SARS-CoV-2 ORF3a or E protein in the sample.

### EXAMPLES

### Example 1. Role of SARS-CoV-2 ORF3a on the NLRP3 Inflammasome

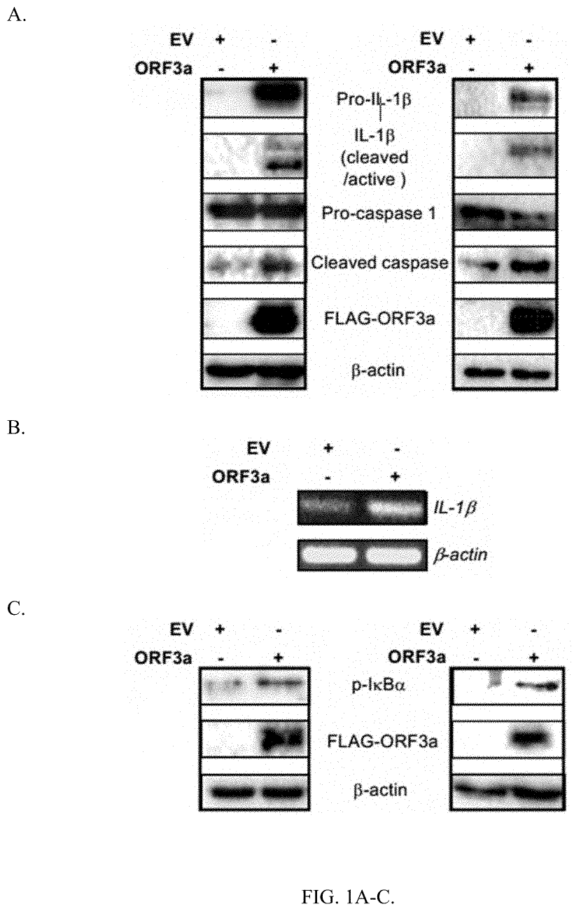

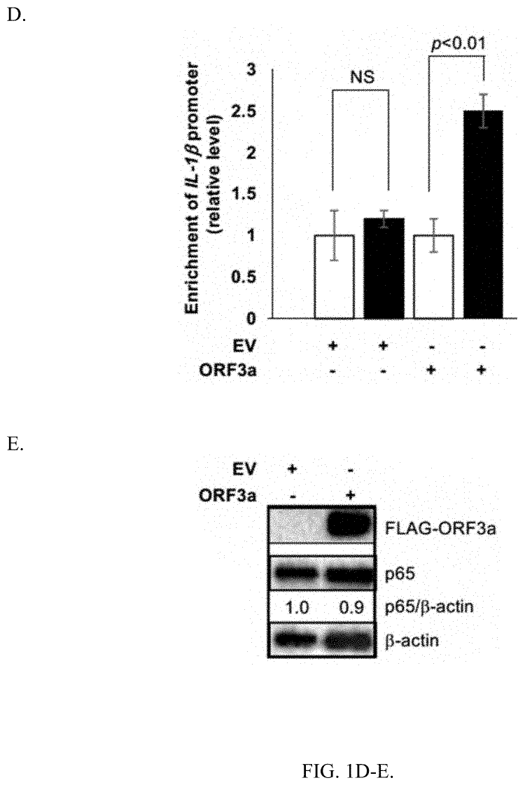

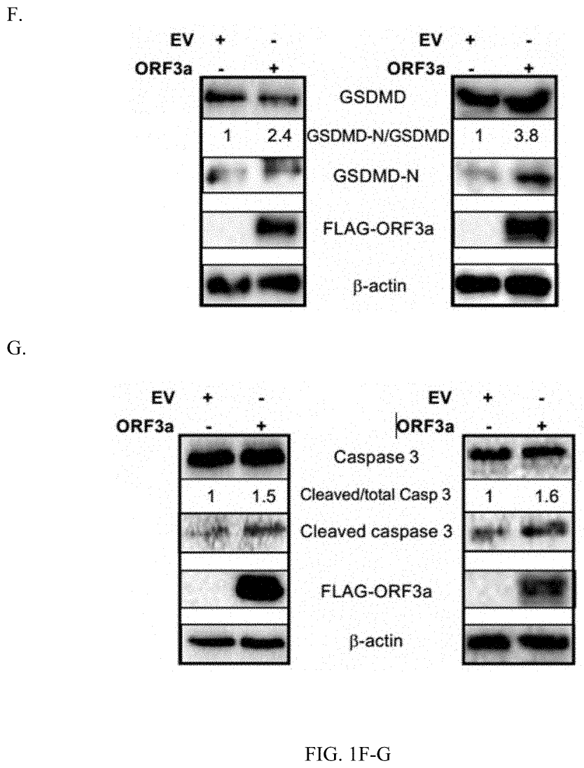

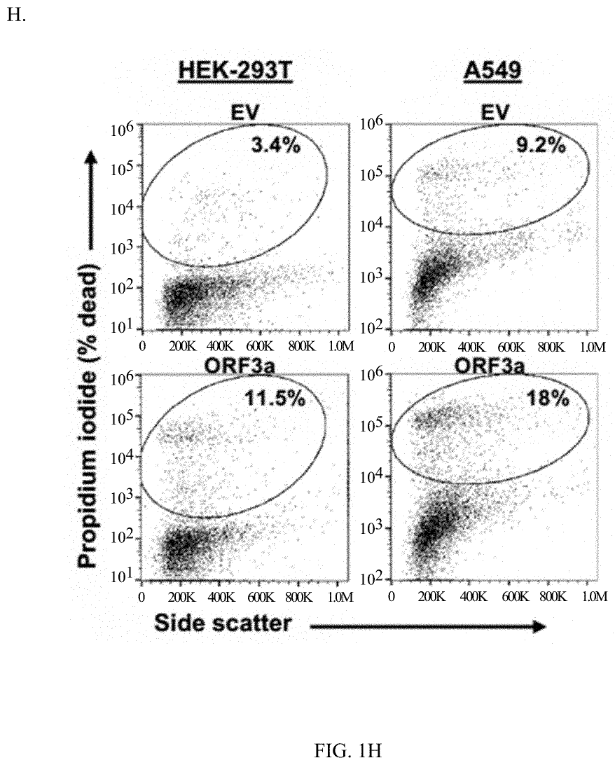

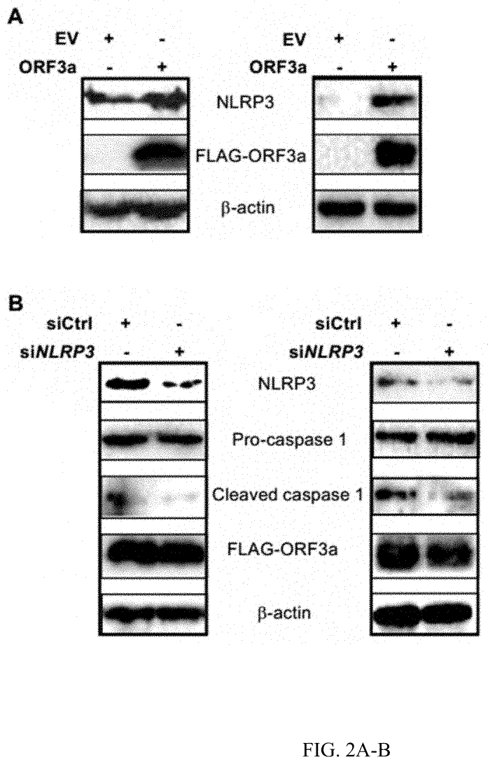

With lung as the predominant site of pathology along with established tropism for kidney, ORF3a was introduced into A549 cells and for comparison, kidney origin HEK-293T cells. Induction of pro-IL-1β was observed in both cell types, consistent with priming of the inflammasome. The mature form of IL-1β represents an important effector cytokine in inflammation with pleiotropic effects causing lymphocyte activation, immune cell recruitment, inflammation-associated programed cell death (pyroptosis), and manifestations of increased pain sensitivity and fever (Dinarello C A. Immunol Rev 281, 8-27 (2018)). Compared to empty vector-exposed cells, ORF3a also increased the levels of the cleaved (active) form of the pro-inflammatory caspase, caspase 1, as well as the cleaved form of the caspase 1 substrate, pro-IL-1β, indicating activation of the inflammasome (FIG. 1A). Priming by ORF3a resulted from NFκB-mediated expression of IL-1β message (FIG. 1B) as indicated by increased IκBα phosphorylation and enrichment of p65 at the IL-1β promoter in ORF3a-exposed cells (FIG. 1C-E). Expression of ORF3a also caused cleavage/activation of Gasdermin, the pyroptosis-inducing caspase 1-substrate, indicated by an increase in the N-terminal fragment of Gasdermin (FIG. 1F). This was accompanied by ORF3a-mediated increased cleavage/activation of caspase 3 and cell death, likely secondary to both pyroptosis and apoptosis (FIG. 1G-H).

### Example 2. ORF3a-Mediates Activation of the Inflammasome

Inflammatory responses, initiated by the expression and release of activated pro-inflammatory cytokines such as IL-1β, IL-18 and others, is triggered by the recognition of a variety of danger signals leading to priming, assembly, and activation of multimeric complexes known as inflammasomes. The most promiscuous of these is the NLRP3 inflammasome, central in atherosclerosis, obesity, and diabetes (all comorbidities associated with severe COVID-19). The inflammatory response is likewise central to severe COVID-19 disease (Farag N S et al. Int J Biochem Cell Biol 122, 105738 (2020) and Fung S Y et al. Emerg Microbes Infect 9, 558-570 (2020)). SARS-like betacoronaviruses encode at least two viroporins (ORF3a and E), both important for pathogenesis, and deletion of either from SARS-CoV blocks activation of the NLRP3 inflammasome (Nieto-Torres J L et al. PLoS Pathog 10, e1004077 (2014) and Siu K L et al. FASEB J33, 8865-8877 (2019)). ORF3a and I also have important functions in the virus's life cycle: E is an envelope protein while ORF3a modulates virus release from the cell. Both proteins form ion channels/pores (Farag N S et al. Int J Biochem Cell Biol 122, 105738 (2020)) and both have been shown to cause K⁺ efflux, a key step in NLRP3 inflammasome activation.

Elevated expression of NLRP3 defines a key step in priming the inflammasome. ORF3a and E are highly conserved among all SARS-like betacoronaviruses, and mutations in key residues of SARS-CoV render them incapable of activating the NLRP3 inflammasome (Farag N S et al. Int J Biochem Cell Biol 122, 105738 (2020); Fung S Y et al. Emerg Microbes Infect 9, 558-570 (2020); and Pervushin K et al. PLoS Pathog 5, e1000511 (2009)).

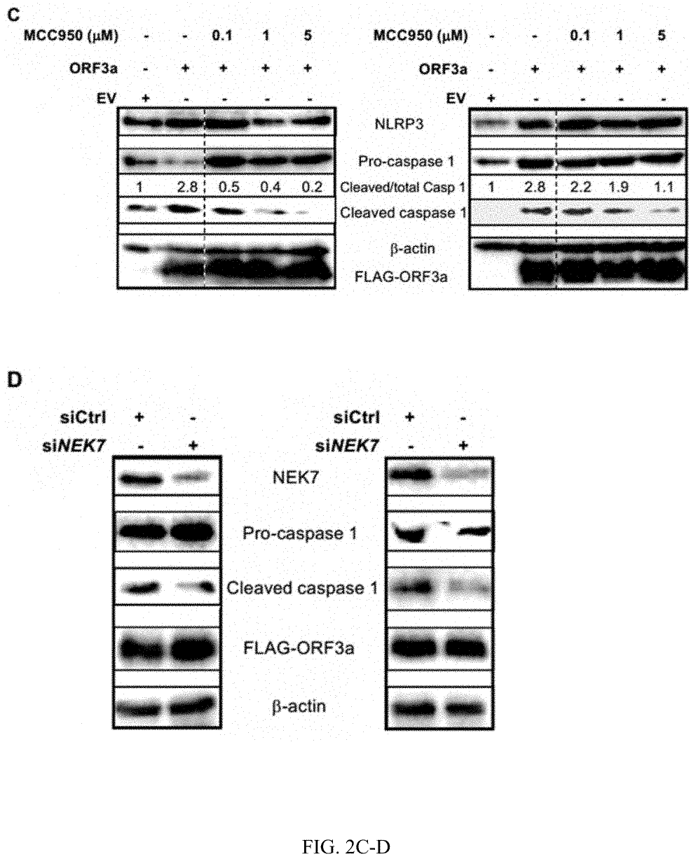

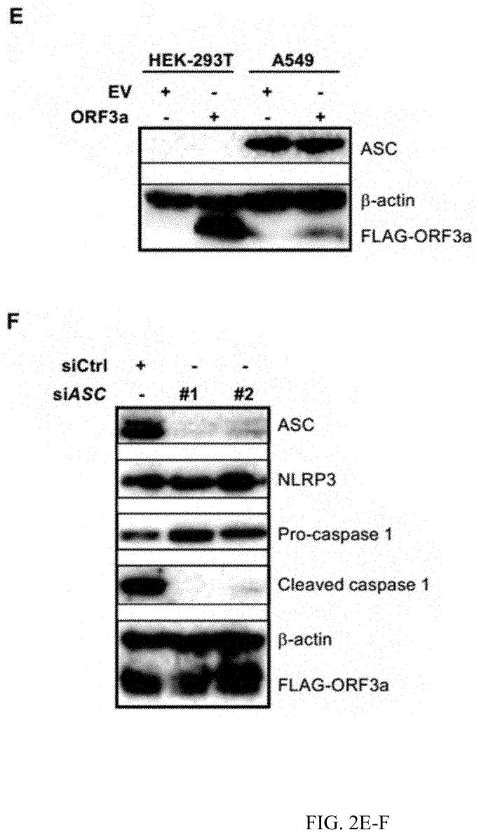

SARS-CoV-2 ORF3a alone is sufficient for priming the NLRP3 inflammasome, illustrated by increased expression of NLRP3 in ORF3a transfected human lung A549 cells (FIG. 2A). Knockdown of NLRP3 curbed ORF3a-directed caspase 1 cleavage (FIG. 2B), indicating activation of the NLRP3 inflammasome by ORF3a. Further, MCC950, a selective small molecule inhibitor that binds to the NACHT domain of NLRP3 and curtails its activation by blocking ATP hydrolysis (Coll RC et al., MCC950 directly targets the NLRP3 ATP-hydrolysis motif for inflammasome inhibition. Nat Chem Biol 15, 556-559 (2019)), also blocked ORF3a-mediated activation of the inflammasome in low micromolar concentrations (FIG. 2C). Moreover, with the NIMA-related kinase NEK7 recently linked to NLRP3 activation (He Y et al., NEK7 is an essential mediator of NLRP3 activation downstream of potassium efflux. Nature 530, 354-357 (2016)), we also depleted NEK7 and found that ORF3a was impaired in its ability to cause cleavage of caspase 1 (i.e., it was unable to activate the inflammasome (FIG. 2D). The NLRP3 inflammasome is activated by a variety of cell-extrinsic and -intrinsic stimuli that trigger the assembly of the inflammasome machinery wherein NLRP3 oligomerizes with the adaptor protein ASC (Apoptosis-associated speck-like protein containing a CARD) leading to recruitment of pro-caspase 1 which is then activated by proximity-induced intermolecular cleavage. Given ORF3a-mediated inflammasome activation in HEK-293T cells that lack ASC (FIG. 2E), we asked if ORF3a activated the inflammasome solely in an ASC-independent manner. We found that ORF3a′s ability to activate pro-caspase 1 was substantially impaired upon depletion of ASC in A549 cells (FIG. 2F), supporting the idea that ORF3a activates the inflammasome in ASC-dependent and -independent ways.

Summary: SARS-CoV-2 ORF3a alone was sufficient for priming the NLRP3 inflammasome, illustrated by increased expression of NLRP3 in ORF3a transfected human lung A549 cells (FIG. 2A).

### Example 3. ORF3a Forms Inward-Rectifier K⁺ Channels in the Cell Membrane

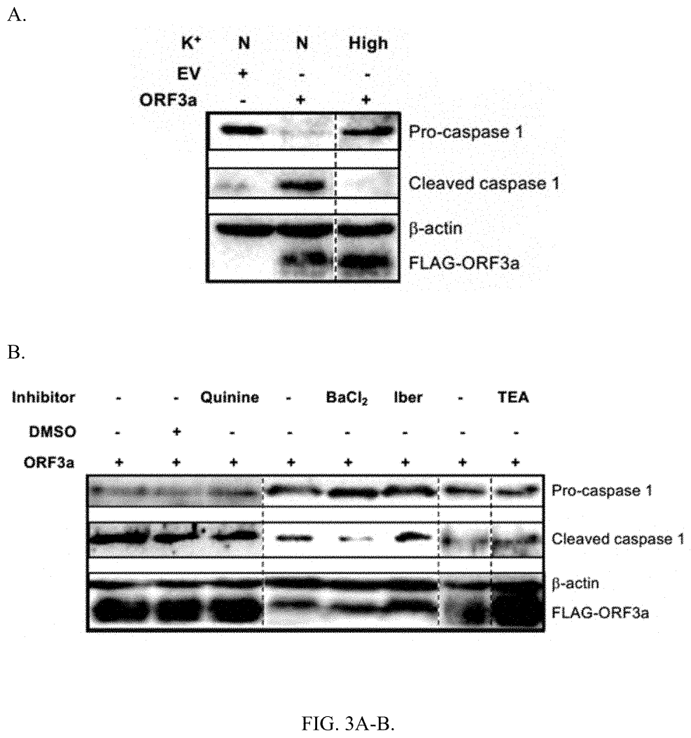

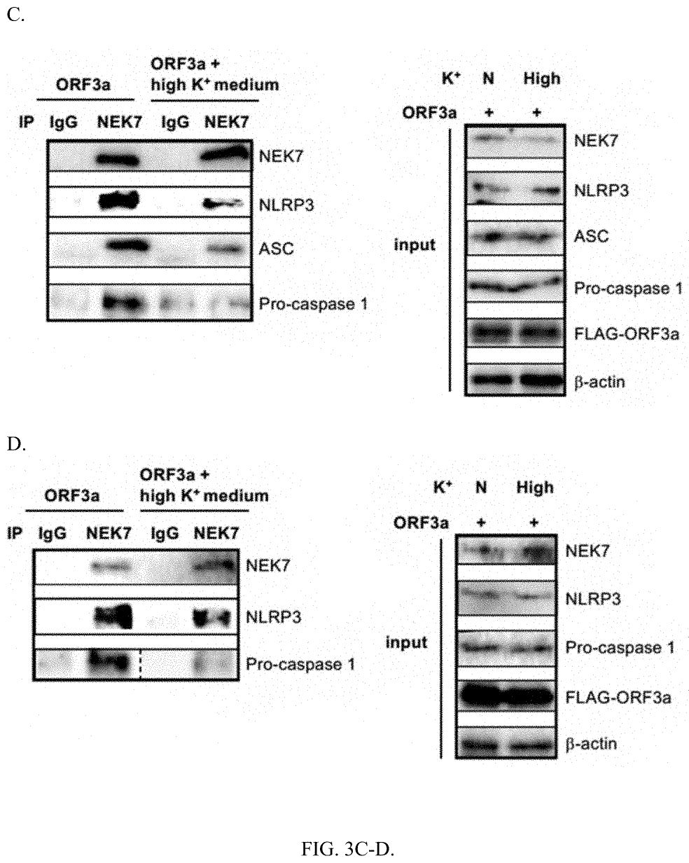

With NEK7 a key mediator of NLRP3 activation downstream of potassium efflux, and efflux of potassium ions a known trigger of the NLRP3 inflammasome, particularly by ion channel-inducing viroporins (Farag NS et al. Int J Biochem Cell Biol 122, 105738 (2020); He Y et al. Nature 530, 354-357 (2016); Chen IY et al. Front Microbiol 10, 50 (2019)), we investigated the effect of blocking potassium efflux by raising the extracellular concentration of K⁺and found that ORF3a-mediated caspase 1 cleavage was abrogated (FIG. 3A), indicating that ORF3a requires K⁺ efflux to activate the inflammasome. To identify the type of K⁺ channel formed by ORF3a, we employed known pharmacologic inhibitors including quinine, barium, iberiotoxin, and tetraethylammonium to block two-pore domain K⁺channels, inward-rectifier K⁺ channels, large conductance calcium-activated K⁺channels, and voltage gated K⁺ channels, respectively (Di A et al. Immunity 49, 56-65 e54 (2018)). Mimicking the ability of barium to block the release of SARS-CoV virions (Lu W et al. Proc Natl Acad Sci USA 103, 12540-12545 (2006)) and supporting the finding in FIG. 3A, barium was able to curb CoV-2 ORF3a-mediated activation of caspase 1, indicating that ORF3a forms inward-rectifier K+ channels in the cell membrane (FIG. 3B). Furthermore, restricting K⁺ efflux impaired ORF3a's ability to trigger assembly of both ASC-dependent and -independent NLRP3 inflammasomes (FIGS. 3C and D).

### Example 4. Sequence Alignment of ORF3a from Coronavirus Isolates

Alignment of ORF3a sequences from SARS-CoV-2 isolates obtained from Asia, Europe, Middle-East, Russia, and North and South America between December 2019 and June 2020 as well as other bat CoVs and SARS-CoV revealed that two out of three key cysteine residues in the cysteine-rich domain (residues 127-133), known to be essential for forming K⁺channels (Chen IY et al. Front Microbiol 10, 50 (2019)), were conserved in all isolates of CoV-2 (FIG. 4A). In contrast, cysteine 127 was replaced by leucine in all CoV-2 isolates. We also observed a similar switch from cysteine to valine at position 121 but a switch from asparagine to cysteine at position 153 in all CoV-2 isolates. Introducing single point mutations at positions 127, 130, and 133 of CoV-2 ORF3a impaired its ability to activate the inflammasome, supporting the need for not only the two conserved cysteines at positions 130 and 133 but also that of the newly acquired leucine at position 127 of CoV-2 ORF3a (FIG. 4B).While SARS-CoV-2 ORF3a has diverged from its homolog in other coronaviruses including SARS-CoV, the domain essential for forming ion channels for virus release has remained remarkably well conserved during the pandemic, thereby also maintaining its ability to activate the inflammasome.

The evidence indicates that viroporin plays a role in release of SARS-CoV-2 from infected cells and is also able to prime and activate the NLRP3 inflammasome, the machinery responsible for much of the inflammatory pathology in severely ill patients. ORF3a's indispensability to the virus's life cycle makes it an important therapeutic candidate. Moreover, while different from its relatives in other CoVs, the high conservation of the newly divergent SARS-CoV-2 ORF3a across isolates from several continents combined with our observation that even single point mutations at key residues are poorly tolerated, argues against rapid emergence of resistance phenotypes. Thus, targeting ORF3a can be used to block virus release and inflammation. ORF E (E) is another SARS-CoV-2 viroporin that may contribute to the inflammatory response by similar or related mechanisms.

### Example 5. Cloning and Characterization of Antibodies from Individuals that Have Recovered from COVID-19.

As shown in FIG. 5A flow cytometry can be used to identify primary B cells from a human donor using the B cell specific markers CD19 and CD20. LCLs (Lymphoblastoid cell lines; i.e., EBV-transformed B cells) proliferate as transformed B cell clusters (FIG. 5B-C) and display nearly unlimited proliferating capacity (FIG. 5D showing results from an experiment characterizing the inhibitory effects of the AP-1 transcription factor inhibitor drug, T-5224). Isolation of primary B cells and establishment of immortalized B cell lines from individuals who have recovered from COVID-19 is used to provide a repository of antibody producing cells from which to mine antibodies targeting SARS-CoV-2.

Primary B lymphocytes are obtained from healthy convalescent patients who have recovered from SARS-CoV-2 infection. The isolated B lymphocytes (B cells) are then immortalized using Epstein-Barr virus (EBV), a B-lymphocyte transforming virus. Immortalized B lymphocytes are screened to identify cells having affinity for coronavirus viroporins ORF31 and/or E. The identified B lymphocytes can be activated and used as a source of human anti-viroporin antibodies. Alternatively, the anti-viroporin antibody genes can be cloned from the identified B lymphocytes to generate recombinant nucleic acids encoding anti-viroporin antibodies. The recombinant anti-viroporin antibody genes can be expressed in cells suitable for expression and in vitro production of antibodies. Antibodies produced from the immortalized B lymphocytes or cells expressing the recombinant antibodies can be screened for efficacy in treating or preventing SARS-CoV-2 infection or COVID-19 or one or more symptoms associated with SARS-CoV-2 infection or COVID-19.

Humanized mouse mAb approaches, including traditional murine hybridoma technologies do not fully recapitulate the humoral immune response of a human, and can elicit immune reactions against murine components, thereby limiting their therapeutic value. Likewise, convalescent plasma varies in antibody content, is heterogeneous, and brings inherent risks of inflammatory reactions and extraneous agent infections. Producing human recombinant antiviral antibodies for primary and immortalized B cells will provide a valuable source of antiviral therapeutics.

### Example 6. High-Throughput Single Cell Partitioning Using the 10× Genomics Inc. Platform and the LIBRA-Seq (Linking B Cell Receptor to Antigen Specificity Through Sequencing) Approach

Viroporin blocking antibody genes are identified from both primary and Epstein-Barr virus (EBV) immortalized memory B cells from healthy donors who have recovered from COVID-19. The 10× Genomics cell partitioning platform indexes each cell to one of hundreds of thousands of sequencing bar codes bound to gel beads (FIG. 6). LIBRA-seq likewise tags antigens of interest with unique sequence barcodes (FIG. 6). Using this system, it is possible to massively parallel sequence hundreds of thousands of individual B cell surface IgG genes encoding antibodies with viroporin-specific binding. Through quantitative analysis, the most abundant and specific IgG gene candidates are resolved.

LIBRA-seq is a technology for high-throughput mapping of paired heavy- and light-chain B cell receptor sequences to their cognate antigen specificities (Setliff I et al. Cell 179, 1636-1646 e1615 (2019).

The B cell receptor (BCR) is a transmembrane protein on the surface of a B cell composed of an immunoglobulin molecule that forms a type 1 transmembrane receptor protein. Activation by an antigen binding to its receptor, results in the B cell producing antibodies.

High-throughput analysis of primary and immortalized B cells provides IgG sequenced candidates for recombinant immunotherapeutics. The cells are mined for recombinant human antibodies capable of interfering with virus replication and/or NLRP3 inflammasome activation. EBV transformed B cell lines can also be enriched by SARS-CoV-2 antigen binding and separation (such as magnetic separation) to provide inexhaustible sources of monoclonal antibody producing cells or polyclonal antibody producing populations of cells. In some embodiments, combinations of immortalized B cells producing different anti-viroporin antibodies are used. Such combinations can provide mixtures of antibodies with different binding specificities, avidities, and antiviral or anti-inflammatory functions.

B cells obtained from convalescent individuals are be divided into two portions, for immortalization by infection with EBV and for direct staining with ORF3a or E recombinant viroporins.

For establishing immortalized B cell lines, enriched primary memory B cells are infected with the B95-8 strain of EBV in the presence CpG (Hui-Yuen J et al. Methods Mol Biol 1131, 183-189 (2014) and Corti D et al. Microbiol Spectr 2 (2014)). Transformations are assessed microscopically by the formation of cell clusters and by cell proliferation assays. Immortalized B cell lines serve as seed for the production of protective antibodies and/or reagent antibodies targeting the complete SARS-CoV-2 or SARS-CoV-2 ORF3a and/or E proteins, as well as for additional cloning of genes encoding protective antibodies. As needed, B cell lines are induced into antibody secreting plasma cells as described in Jourdan M. et al. Blood 114, 5173-5181 (2009) and Jourdan M et al. J Vis Exp, Jan 20; (143) (2019).

Primary and immortalized memory B cells (CD19⁺/CD27⁺and/or CD20⁺/CD27⁺) are stained with recombinant ORF3a or E proteins/peptides each conjugated to a unique 10× Genomic compatible oligonucleotide barcode as well as to the fluorophore phycoerythrin (PE). For bulk fluorescence activated cell sorting ahead of 10× Genomics partitioning and LIBRA-seq, B cells are simultaneously stained with a cocktail consisting of anti-IgG-FITC, LiveDead-V500, and PE labeled DNA-barcoded antigens.

Complete and various portions of ORF3a and E genes from the UF-1 strain of SARS-CoV-2 are cloned into plasmids. The plasmids are then expressed in bacteria as 6×His tagged peptides. The expressed 6×His tagged peptides are then purified by Nickel NTA column purification. Biotinylated peptides spanning overlapping regions of ORF3a and E are also synthesized. Recombinant protein antigens are directly conjugated to unique DNA oligonucleotide barcodes (one for each antigen) using the Solulink Protein-Oligonucleotide Conjugation Kit (TriLink, Inc).

Single cell partitioning of antigen bound B cells is performed on a Chromium Controller (10× Genomics Inc.) followed by oligo dT primed cDNA synthesis and immunoglobulin heavy and light chain VDJ region sequencing on an Illumina NextSeq as previously described for LIBRA-seq (Setliff, I. et al. Cell 179, 1636-1646 e1615, (2019)). Paired end sequence reads are quality score filtered. Reads without a cell barcode and reads containing multiple antigen barcodes are eliminated. Reads with two barcodes (one for antigen and one for cell) are retained for further analysis. Heavy and light chain (Igk or Igic) VDJ sequences are assembled using 10× Genomics Cell Ranger Analysis Pipelines.

Heavy and light chain variable sequences are commercially synthesized and cloned separately into Invivogen Inc. Eukaryotic expression vectors containing human Ig constant regions for the heavy chain (pFUSEss-CHIghG1) and light chains (Igλ or Igκ: pFUSE2ss-CLIg-hl2 and pFUSE2ss- CLIg-hk, respectively).

Plasmids encoding recombinant human heavy and light chain IgG are co-transfected into HEK293T cells or other suitable cells in triplicate by lipofection for expression. Cell culture media are assayed 72 h later for the expression of IgG by anti-human IgG ELISA using plates coated with cognate recombinant antigens derived from ORF3a or E. Likewise, media derived from recombinant IgG for each clone are tested for their ability to recognize viroporins expressed by transfection in human kidney 293T cells at 24 h as compared to empty vector control transfections by immunofluorescence assay (IFA).

### Example 7. Identification of Rrecombinant Human-Derived mAbs Targeting Viroporins that Interfere with Virus Replication and/or NLRP3 Inflammasome Activation

Induction of NLRP3 and intracellular pro-IL-1β (FIGS. 2 and 3) provide functional assays for screening the effectiveness of antibodies targeting the SARS-CoV-2 viroporins in blocking or inhibiting inflammasome activation. Cloned antibodies are also assessed for their ability to block or inhibit K⁺ efflux leading to inflammasome activation.

Antibodies demonstrating high signal in antigen-specific ELISA and/or IFA against cognate antigens are functionally tested for their ability to block inflammasome activation. Anti-viroporin antibodies are assayed for inhibition of NRLP3 inflammasome using a flow cytometric test for pro-IL-1β expression and/or an IL-1β ELISA assessing blockade of K⁺ efflux. This screen, which can be adapted for rapid screening, provides for selection of top candidates ahead of functional tests in Bio Safety Level 3 (BSL3) using wildtype SARS-CoV-2.

Human lung A549 cells and kidney 293T cells are transfected with viroporins ORF3a or E and incubated in the presence or absence of candidate recombinant antibodies. At 24 h post transfection, cells are fixed, permeabilized and, stained for NLRP3 and analyzed by flow cytometry. The absence and/or decrease in cells expressing NLRP3 as compared to viroporin-antibody minus controls indicates activity of recombinant antibodies in blocking priming of the NLRP3 inflammasome. A similar flow cytometric test for the expression level of IL-1β is used to further evaluate the activity of recombinant antibodies in blocking inflammasome activation (downstream of priming). To assess K⁺ ion channel blocking activities, ORF3a or E is expressed in A549 cells. Cells are assayed by flow cytometry for pro-IL10 expression and media by ELISA for mature/cleaved IL-1β in the presence or absence of recombinant antibody, thereby assessing the ability of the antibodies to block K⁺ efflux leading to inflammasome activation.

### Example 8. Activity Against SARS-CoV-2 Infection

Titering antiviral activities against wild type SARS-CoV-2 isolates in a Biosafety Level 3 (BSL3) environment is used to test efficacy of the antibodies in inhibiting SARS-CoV-2 infection. Antibodies are tested for their ability to interfere with primary infection by SARS-CoV-2. Antibodies are also tested on virus-infected primary lung epithelial cells and/or stable ACE2-expressing human lung A549 cells, using at least two different virus isolates, for their ability to reduce cytopathic effects (microscopically), virus titer (plaque assay/RT-qPCR), and/or inflammasome activation via mature cleaved IL-1β production (by ELISA).

### REFERENCES

Setliff I et al. High-Throughput Mapping of B Cell Receptor Sequences to Antigen Specificity. Cell 179, 1636-1646 e1615 (2019).

Huang C et al. Clinical features of patients infected with 2019 novel coronavirus in Wuhan, China. Lancet 395, 497-506 (2020).

Li G et al. Therapeutic options for the 2019 novel coronavirus (2019-nCoV). Nat Rev Drug Discov 19, 149-150 (2020).

Zhang L et al. Potential interventions for novel coronavirus in China: A systematic review. J Med Virol 92, 479-490 (2020).

Ye M et al. Treatment with convalescent plasma for COVID-19 patients in Wuhan, China. J Med Virol (2020).

Chen L et al. Convalescent plasma as a potential therapy for COVID-19. Lancet Infect Dis 20, 398-400 (2020).

Mulangu S et al. A Randomized, Controlled Trial of Ebola Virus Disease Therapeutics. N Engl J Med 381, 2293-2303 (2019).

Seghatchian J et al. Pathogen-reduction systems for blood components: the current position and future trends. Transfus Apher Sci 35, 189-196 (2006).

Wu Z et al. Characteristics of and Important Lessons From the Coronavirus Disease 2019 (COVID-19) Outbreak in China: Summary of a Report of 72314 Cases From the Chinese Center for Disease Control and Prevention. JAMA 323(13):1239-1242 (2020).

Alhawiti N M et al. TXNIP in Metabolic Regulation: Physiological Role and Therapeutic Outlook. Curr Drug Targets 18, 1095-1103 (2017).

Chen W et al. Activation of the TXNIP/NLRP3 inflammasome pathway contributes to inflammation in diabetic retinopathy: a novel inhibitory effect of minocycline. Inflamm Res 66, 157-166 (2017).

Jin Y et al. Novel Insights Into the NLRP 3 Inflammasome in Atherosclerosis. J Am Heart Assoc 8, e012219 (2019).

Rheinheimer J et al. Current role of the NLRP3 inflammasome on obesity and insulin resistance: A systematic review. Metabolism 74, 1-9 (2017).

Stienstra R et al. Inflammasome is a central player in the induction of obesity and insulin resistance. Proc Natl Acad Sci USA 108, 15324-15329 (2011).

Dixit V D, Nlrp3 inflammasome activation in type 2 diabetes: is it clinically relevant? Diabetes 62, 22-24 (2013).

Grant R W et al. Mechanisms of disease: inflammasome activation and the development of type 2 diabetes. Front Immunol 4, 50 (2013).

Zhang X et al. Reactive oxygen species-induced TXNIP drives fructose-mediated hepatic inflammation and lipid accumulation through NLRP3 inflammasome activation. Antioxid Redox Signal 22, 848-870 (2015).

Feldstein LR et al. Multisystem Inflammatory Syndrome in U.S. Children and Adolescents. N Engl J Med 383, 334-346 (2020).

DeDiego M L et al. A severe acute respiratory syndrome coronavirus that lacks the E gene is attenuated in vitro and in vivo. J Virol 81, 1701-1713 (2007).

DeDiego M L et al. Coronavirus virulence genes with main focus on SARS-CoV envelope gene. Virus Res 194, 124-137 (2014).

DeDiego M L et al. Pathogenicity of severe acute respiratory coronavirus deletion mutants in hACE-2 transgenic mice. Virology 376, 379-389 (2008).

Farag N S et al., Viroporins and inflammasomes: A key to understand virus-induced inflammation. Int J Biochem Cell Biol 122, 105738 (2020).

Castano-Rodriguez C. et al. Role of Severe Acute Respiratory Syndrome Coronavirus Viroporins E, 3a, and 8a in Replication and Pathogenesis. mBio 9 (2018).

Chen I Y et al. Severe Acute Respiratory Syndrome Coronavirus Viroporin 3a Activates the NLRP3 Inflammasome. Front Microbiol 10, 50 (2019).

Fung S Y et al. A tug-of-war between severe acute respiratory syndrome coronavirus 2 and host antiviral defense: lessons from other pathogenic viruses. Emerg Microbes Infect 9, 558-570 (2020).

Nieto-Torres J L et al. Severe acute respiratory syndrome coronavirus envelope protein ion channel activity promotes virus fitness and pathogenesis. PLoS Pathog 10, e1004077 (2014).

Siu K L et al. Severe acute respiratory syndrome coronavirus ORF3a protein activates the NLRP3 inflammasome by promoting TRAF3-dependent ubiquitination of ASC. FASEB J33, 8865-8877 (2019).

Pervushin K et al. Structure and inhibition of the SARS coronavirus envelope protein ion channel. PLoS Pathog 5, e1000511 (2009).

Dinarello C A. Overview of the IL-1 family in innate inflammation and acquired immunity. Immunol Rev 281, 8-27 (2018).

Frey T R et al. Nascent Transcriptomics Reveal Cellular Prolytic Factors Upregulated Upstream of the Latent-to-Lytic Switch Protein of Epstein-Barr Virus. J Virol 94, (2020).

Hui-Yuen J et al. Human B cell immortalization for monoclonal antibody production. Methods Mol Biol 1131, 183-189 (2014).

Xu H et al. Novel replisome-associated proteins at cellular replication forks in EBV-transformed B lymphocytes. PLoS Pathog 15, e1008228 (2019).

Corti D et al. Efficient Methods To Isolate Human Monoclonal Antibodies from Memory B Cells and Plasma Cells. Microbiol Spectr 2 (2014).

Jourdan M et al. An in vitro model of differentiation of memory B cells into plasmablasts and plasma cells including detailed phenotypic and molecular characterization. Blood 114, 5173-5181 (2009).

Jourdan M et al. In Vitro Differentiation Model of Human Normal Memory B Cells to Long-lived Plasma Cells. J Vis Exp, January 20; (143) (2019).

Morris, S. B. et al. Case series of Multisystem Inflammatory Syndrome in Adults associated with SARS-CoV-2 infection—United Kingdom and United States, March-August 2020. Morbidity and Mortality Weekly Report (MMWR) 69, 1450-1456 (2020)

1. A method of generating human antibodies capable of blocking one or more functions of a coronavirus viroporin comprising:
(a) obtaining primary human B-lymphocytes from a subject that has recovered from coronavirus infection;
(b) identifying one or more primary human B-lymphocytes having surface IgG having affinity for the viroporin;
(c) immortalizing the one or more B-lymphocytes identified as having surface IgG having affinity for the viroporin; and
(d) producing anti-viroporin antibodies from the one or more immortalized B-lymphocytes.

2. A method of generating human antibodies capable of blocking one or more functions of a coronavirus viroporin comprising:
(a) obtaining primary human B-lymphocytes from a subject that has recovered from coronavirus infection;
(b) immortalizing the B-lymphocytes from step (a);
(c) identifying one or more immortalized B-lymphocytes having surface IgG having affinity for the viroporin; and
(d) producing anti-viroporin antibodies from the one or more immortalized B-lymphocytes identified as having surface IgG having affinity for the viroporin.

3. A method of generating human antibodies capable of blocking one or more functions of a coronavirus viroporin comprising:
(a) obtaining primary human B-lymphocytes from a subject that has recovered from coronavirus infection;
(b) identifying one or more primary human B-lymphocytes having surface IgG having affinity for the viroporin,
(c) generating one or more recombinant nucleic acids encoding anti-viroporin antibodies or antigen binding fragments thereof from the one or more B-lymphocytes identified having surface IgG having affinity for the viroporin;
(d) generating one or more cell lines expressing the one or more recombinant nucleic acids; and
(e) producing anti-viroporin antibodies from the one or more cell lines expressing the one or more recombinant nucleic acids.

4. The method of claim 3, further comprising
immortalizing the B-lymphocytes prior to
identifying the one or more immortalized B-lymphocytes having surface IgG having affinity for the viroporin.

5. The method of claim 3 wherein the coronavirus is a betacoronavirus.

6. The method of claim 5, wherein the betacoronavirus is SARS-CoV-2.

7. The method of claim 3, wherein the viroporin is an ORF3a protein or an E protein.

8. A method of treating or preventing coronavirus infection in a subject comprising administering to the subject an effective amount of the antibodies of claim 3.

9. The method of claim 8, wherein treating coronavirus infection comprises treating or preventing one or more symptoms associated with coronavirus disease or infection.

10. The method of claim 9, wherein the symptom associated with coronavirus disease or infection comprises an inflammatory response.

11. The method of claim 10, wherein the inflammatory response comprises a cytokine storm, an inflammasome-associate response, an IL-Iβ-associated response, an NLRP3-associated response, Multisystem Inflammatory Syndrome in Children, or Multi system Inflammatory Syndrome in Adults.

12. The method of claim 10, wherein the subject has defective type I interferon immunity, has a chronic disease marked by pro-inflammatory conditions characterized by activation of the NLRP3 inflammasome, suffers from a metabolic disturbance, is immunocompromised, has diabetes, has atherosclerosis, and/or is obese.

13. The method of claim 12, wherein the metabolic disturbance comprises hypokalemia.

14. The method of claim 9, wherein the coronavirus disease is COVID-19.

15. The method of claim 8, wherein the coronavirus infection is SARS-CoV-2 infection.

16. The method of claim 8, further comprising administering to the subject an effective dose of an additional anti-coronavirus antibody therapy, a convalescent plasma therapy, an anti-inflammation therapy, or an inflammasome therapy.

17. A method of diagnosing SARS-CoV-2 infection comprising analyzing (a) obtaining a sample from a subject; and (b) using an antibody of claim 7 to detect the presence or absence of a SARS-CoV-2 ORF3a or E protein in the sample.


SEQUENCE LISTING (23 sequences)

SEQ ID NO: 1 - protein, 80 aa, SARS-CoV-2
CNLLLLFVTVYSHLLLVAAGLEAPFLYLYALVYFLQSINFVRIIMRLWLCWKCRSKNPLLYDANYFLCWHTNCYDYCIPY

SEQ ID NO: 2 - protein, 80 aa, SARS-CoV-2; identical to SEQ ID NO: 1

SEQ ID NO: 3 - protein, 80 aa, SARS-CoV-2; identical to SEQ ID NO: 1

SEQ ID NO: 4 - protein, 80 aa, SARS-CoV-2; identical to SEQ ID NO: 1

SEQ ID NO: 5 - protein, 80 aa, SARS-CoV-2; identical to SEQ ID NO: 1

SEQ ID NO: 6 - protein, 80 aa, SARS-CoV-2
CNLLLLFVTVYSHLLLVAAGLEAPFLYLYALVYFLQSINFVRIIMRLWLCWKCRSKNPLKYDANYFLCWHTNCYDYCIPY

SEQ ID NO: 7 - protein, 80 aa, SARS-CoV-2
CNLLLLFATVYSHLLLVAAGLEAPFLYLYALVYFLQSINFVRIIMRLWLCWKCRSKNPLLYDANYFLCWHTNCYDYCIPY

SEQ ID NO: 8 - protein, 80 aa, SARS-CoV-2; identical to SEQ ID NO: 1

SEQ ID NO: 9 - protein, 80 aa, SARS-CoV-2; identical to SEQ ID NO: 1

SEQ ID NO: 10 - protein, 80 aa, SARS-CoV-2; identical to SEQ ID NO: 1

SEQ ID NO: 11 - protein, 80 aa, SARS-CoV-2; identical to SEQ ID NO: 1

SEQ ID NO: 12 - protein, 80 aa, SARS-CoV-2; identical to SEQ ID NO: 1

SEQ ID NO: 13 - protein, 80 aa, SARS-CoV-2
CNLLLLFVTVYSHLLLVAAGLEAPFLYLYALVYFLQSINFVRIIMRLWLCWKCRSKNPLLYDSNYFLCWHTNCYDYCIPY

SEQ ID NO: 14 - protein, 80 aa, SARS-CoV-2; identical to SEQ ID NO: 1

SEQ ID NO: 15 - protein, 80 aa, SARS-CoV-2; identical to SEQ ID NO: 1

SEQ ID NO: 16 - protein, 80 aa, SARSr-CoV
CNLLLLFLTVYSHLLLLAGGLEATFLFLYALAYCLQTVNFVRIIMRFWLCWKCRSKNPLLYDANYFLCWHTNCYDYCIPY

SEQ ID NO: 17 - protein, 80 aa, SARSr-CoV; identical to SEQ ID NO: 1

SEQ ID NO: 18 - protein, 80 aa, SARSr-CoV
CNLLLLFVIVYSHLLLVAAGMEAQFLYLYALIYFLQCINACRIIMRCWLCWKCRSKNPLLYDANYFVCWHTNNYDYCIPY

SEQ ID NO: 19 - protein, 80 aa, SARSr-CoV; identical to SEQ ID NO: 18

SEQ ID NO: 20 - protein, 80 aa, SARSr-CoV
CNLMLLFVIVYSHLLLLAACMEAQFLYIYALIYILQIVSFCRFIMRCWLCWKCRSKNPLLYDANYFVCWHTNNYDYCIPY

SEQ ID NO: 21 - protein, 80 aa, SARSr-CoV
CNLVLLIALVYSHLLLVAAGLEAPFLYLLALIYFLQCVVFGRLLVRCWLCWKCRSKNPLIYDSNYFVCWHTHNHDYCIPY

SEQ ID NO: 22 - protein, 80 aa, SARSr-CoV
CNLMLLFVIVYSHLLLLAACMEAQFLYIYALIYILQIVSFCRIIMRCWLCWKCRSKNPLLYDANYFVCWHTNNYDYCIPY

SEQ ID NO: 23 - protein, 80 aa, SARS-CoV
CNLLLLFVIVYSHLLLVAAGMEAQFLYLYALIYFLQCINACRIIMRCWLCWKCKSKNPLLYDANYFVCWHTHNYDYCIPY